(19) United States
(12) Patent Application Publication
(10) Pub. No.: US 2010/0015054 A1
(43) Pub. Date: Jan. 21, 2010

(54) FLUORO-SUBSTITUTED BENZOXAZOLE POLYMETHINE DYES

(75) Inventor: Najeeb Said, Amersham (GB)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE UK LIMITED, Little Chalfont (GB)

(21) Appl. No.: 12/443,246

(22) PCT Filed: Oct. 4, 2007
(86) PCT No.: PCT/GB07/03787

§ 371 Date: Mar. 27, 2009

(30) Foreign Application Priority Data
Oct. 5, 2006 (GB) 0619626.5

(51) Int. Cl. A61K 47/22; C07D 413/06; A61K 49/00; G01N 33/566

(52) U.S. Cl. 424/9.1; 548/217; 548/219; 514/788; 436/501

(57) ABSTRACT

Disclosed are reactive polyfluoro benzoxazole polymethine dyes that are useful for labelling and detecting biological and other materials. The dyes are of formula (I):

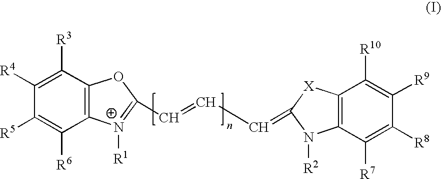

in which X is selected from the group consisting of —O—, —S— and

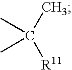

at least one of groups R¹ and R² is the group -L-R^{x} or -L-R^{p}, where L is a linking group, R^{x} is a group suitable for covalent attachment of the dye to a component and R^{p} is a component; and at least one of groups R³, R⁴, R⁵, R⁶, R⁷, R⁸, R⁹ and R¹⁰ comprises fluorine. The use of polymethine dyes substituted by fluorine and having additional substitution by sulphonic acid groups, for labelling biological target molecules results in a labelled product in which there is reduced dye-dye aggregation and improved photostability, compared with cyanine dyes having no such substitutions.

### CROSS-REFERENCE TO RELATED APPLICATIONS

This application is a filing under 35 U.S.C. §371 and claims priority to international patent application number PCT/GB2007/003787 filed Oct. 4, 2007, published on Apr. 10, 2008, as WO 2008/040994, which claims priority to patent application number 0619626.5 filed in Great Britain on Oct. 5, 2006.

The present invention relates to the field of fluorescence labelling reagents, in particular reactive fluoro-benzoxazole polymethine dyes, and the labelling and detection of components labelled with such dyes.

Cyanine dyes are widely used as reagents for fluorescence labelling of biologically important molecules such as proteins, nucleic acids, hormones and drugs. Indeed, cyanine dyes offer a number of advantages over other fluorescent dyes. For example, the excitation and emission spectra of cyanine dyes span the visible and near-infrared spectrum from 450 nm to 800 nm. Furthermore, the cyanine dyes are characterised by having very high extinction coefficients and favourable quantum yields. See for example, U.S. Pat. Nos. 6,048,982, 5,268,486, 5,569,587, (Waggoner, A. S. et al). However, with certain cyanine dye structures there is a tendency towards self-association (or aggregation) leading to fluorescence quenching and a notable hypsochromic wavelength shift in absorbance.

Recently, Waggoner et al (Org. Letters, (2004), 6(6), 909-912) has described a polyfluoro-thiadicarbocyanine dye (Compound (i)) having good photostability in aqueous solvents. The dye exhibited reduced aggregation, enhanced quantum yield and greater resistance to photobleaching when compared with a non-fluorinated analogue.

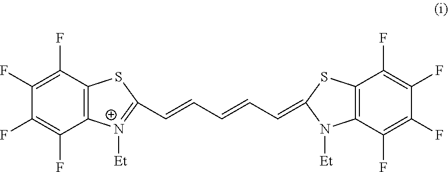

Modification of the indolium ring of a carbocyanine dye at least one of the 3-positions, so as to introduce a reactive group or a conjugated substance has been described in WO 02/26891 (Molecular Probes Inc.). The modified dyes according to WO 02/26891 have also been reported to overcome the tendency of cyanine dyes to self-associate and dye conjugates labelled with the modified dyes are reported to be more fluorescent than conjugates labelled with structurally similar carbocyanine dyes.

Neither of the above documents discloses reactive cyanine dyes containing one and preferably multiple fluoro substituents attached to a benzoxacyanine chromophore as are described herein. The dyes according to the present invention are in addition provided with at least one group suitable for direct covalent labelling of a target material, such as a protein, antibody, nucleic acid, etc. The present invention provides cyanine dye derivatives that have the properties of increased photostability and reduced dye-dye interactions. The dyes are therefore particularly useful in assays involving fluorescence detection where continual excitation is a requirement, for example in kinetic studies, or in microarray analyses where microarray slides may need to be reanalysed over a period of days.

Accordingly, in a first aspect there is provided a compound of formula (I):

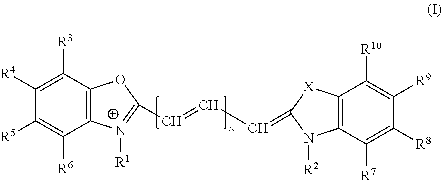

wherein:
X is selected from the group consisting of —O—, —S— and

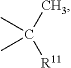

where R¹¹ is CH₃ or —(CH₂)ₖ—SO₃H;
at least one of groups R¹ and R² is the group -L-R^{x} or -L-R^{p}, where L is a linking group having a chain from 1-20 linked atoms selected from the group consisting of carbon, nitrogen and oxygen atoms;
R^{x} is a group suitable for covalent attachment of said compound to a component;
R^{p} is a component;
when either of groups R¹ and R² is not said group -L-R^{x} or -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine.

Preferably, when X is —O— or —S—, groups R³, R⁴, R⁵, R⁶, R⁷, R⁸, R⁹ and R¹⁰ comprises fluorine. R¹⁰ are selected independently from hydrogen, —SO₃H and the group —(CF₂)ₘ—F, where m is 0 or an integer from 1 to 4.

Preferably, n is selected from 1 or 2, i.e. the dyes according to the invention are preferably trimethine or pentamethine dyes, more particularly trimethine dyes.

In one embodiment, X is the group:

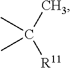

in which case the compound according to the first aspect is of the formula (II):

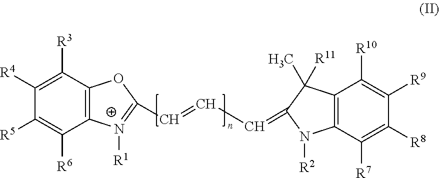

wherein at least one of groups R¹ and R² is the group -L-R^{x} or -L-R^{p}, where L,
R^{x} and R^{p} are hereinbefore defined;
when either of groups R¹ and R² is not said group -L-R^{x} or -L-R^{p}, said remaining group R¹ or R² is selected from C₁-C₄ alkyl and —(CH₂)ₖ—SO₃H;
R¹¹ is CH₃ or —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

In another embodiment, X is —O—, in which case the compound according to the first aspect is of the formula (III):

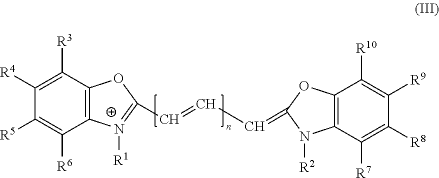

wherein at least one of groups R¹ and R² is the group -L-R^{x} or -L-R^{p}, where L, R^{x} and R^{p} are hereinbefore defined;
when either of groups R¹ and R² is not said group -L-R^{x} or -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

Preferably, in the compounds of formula (I), (II) and (II), at least one of groups R³, R⁴, R⁵, R⁶, R⁷, R⁸, R⁹ and R¹⁰ is fluorine.

The compounds according to the first aspect will suitably include a counter-ion, which may be positive or negative to balance the formal charge (or charges) on the dye chromophore. The nature of the counter-ion is not material to the invention and could be one of many known ions such as NH₄⁺, K⁺, Na⁺, trifluoroacetate (F₃C—CO₂⁻), perchlorate (ClO₄⁻), Br⁻, or I⁻. In the context of the present invention, it is to be understood that the sulphonic acid group (—SO₃H) will also include the sulphonate group (—SO₃⁻), since sulphonate is the ionised form of the parent acid.

The compounds of the first aspect of the invention will suitably comprise at least one, preferably two or more fluorine atoms substituted directly or indirectly onto the dye chromophore. In one embodiment, compounds of formula (I), (II) and (III) may be substituted by a fluorine atom at least one, preferably at least two, and more preferably at least three of the R³, R⁴, R⁵ and R⁶ positions and/or the R⁷, R⁸, R⁹ or R¹⁰ positions. In this embodiment, substitution by one or more fluorine atoms may give rise to symmetric or asymmetric dyes of formula (I). In particularly preferred embodiments, each of the R³, R⁴, R⁵ and R⁶ positions and/or the R⁷, R⁸, R⁹ and R¹⁰ positions are substituted by fluorine. Perfluoro substitution of the dye chromophore has been found to lower dye-dye aggregation, thereby enhancing fluorescence quantum yield and dye photostability (Waggoner, A. et al, loc cit). In a second embodiment, the compounds of formula (I), (II) and (III) may include a perfluoro C₁-C₄ alkyl substituent at one, preferably not more than two of the R³, R⁴, R⁵ or R⁶ positions and/or the R⁷, R⁸, R⁹ or R¹⁰ positions. Any remaining groups R³, R⁴, R⁵, R⁶, R⁷, R⁸, R⁹ and R¹⁰ are selected from H or F. Preferably, the perfluoro C₁-C₄ alkyl substituent is a trifluoromethyl substituent.

Optionally, dyes according to the present invention having 1, 2, 3, 4, or more fluoro groups attached thereto, may be further substituted with one or more sulphonic acid groups attached directly to any of the remaining R³, R⁴, R⁵, R⁶, R⁷, R⁸, R⁹ or R¹⁰ positions unsubstituted by fluoro. Thus, dyes according to the present invention may be substituted directly or indirectly with 1, 2 or 3 sulphonic acid groups. The use of cyanine dyes substituted by fluorine and having additional substitution with two or more sulphonic acid groups for labelling biological molecules results in a labelled product in which there is reduced dye-dye aggregation and improved photostability, compared with cyanines having no such substitutions. The fluorescence emission intensity of a molecule so labelled with the preferred dyes of the present invention increases with the number of covalently attached dyes. Furthermore, N-sulfoalkyl substitution in the heterocyclic ring, in addition to increasing the overall charge on the dye molecule, also adds steric bulk, thereby contributing to a reduction in dye-dye aggregation.

In one embodiment, linking group L links the dye chromophore with R^{x}, a group suitable for covalent attachment of the compound to a component. In a second embodiment, L links the dye directly with R^{p}, that is, the dye is covalently attached and thereby conjugated to a component. In preferred embodiments, the dyes of the present invention will contain one group -L-R^{x} or -L-R^{p} attached to either of the R¹ or R² positions. Remaining group R¹ or R² is selected from C₁-C₄ alkyl, preferably methyl, ethyl or propyl. Alternatively, remaining group R¹ or R² may be —(CH₂)ₖ—SO₃H, where k is an integer from 1 to 10, preferably 3 or 4.

Suitably, L contains from 1-20 linked atoms selected from linear or branched C₁₋₂₀alkyl chains, which may optionally contain one or more linkages selected from —O—, —NR′—, —C(O)—NR′— and phenylene, where R′ is hydrogen or C₁-C₄ alkyl. Preferably, linking group L has from 5 to 12 atoms. More preferably, L is the group —(CH₂)ₚ-Q-(CH₂)ᵣ— where Q is selected from: —CH₂— and —CO—NH—, p is 1-5 and r is 0-5.

In one embodiment, R^{x} is a group that is capable of reacting with a complementary group of a component, with the formation of a covalent linkage between the dye and the component. In this embodiment, the choice of bonding group will depend on the groups that are available on the component to be labelled and, as such, will be well known to those skilled in the art. For example, R^{x} may be a reactive group that can react under suitable conditions with a complementary functional group of a component. Examples of functional groups present in components, such as proteins, peptides, nucleic acids carbohydrates and the like, include hydroxy, amino, sulphydryl, carbonyl (including aldehyde and ketone), carboxylic acid and thiophosphate. Alternatively, R^{x} may be a functional group and the component may contain, or be derivatised to contain a reactive constituent, such that the functional group of the dye may be reacted under suitable conditions with the reactive group of the component. In either case, the component becomes labelled with the dye according to the invention. Suitably, when R^{x} is a reactive group, it is selected from succinimidyl ester, sulpho-succinimidyl ester, isothiocyanate, maleimide, haloacetamide, acid halide, hydrazide, dichlorotriazine and phosphoramidite. Preferably, the reactive group is a succinimidyl ester of a carboxylic acid, an isothiocyanate, a maleimide, a haloacetamide or a phosphoramidite. When R^{x} is a functional group, it is suitably selected from hydroxy, amino, sulphydryl, carbonyl (including aldehyde and ketone), carboxylic acid and thiophosphate. By virtue of these reactive and functional groups the compounds of the present invention may be reacted with and become covalently bound to the component.

Selected examples of reactive groups R^{x} at the R¹ and/or R² positions of the compound according to the invention and the groups with which groups R¹ and/or R² can react to form a covalent linkage are provided in Table 1. In the alternative, R¹ and/or R² may be the functional groups of Table 1 which would react with the reactive groups of a component.

TABLE 1
Examples of reactive groups, functional groups
and covalent linkage formed therefrom

| Reactive Group | Functional Group | Covalent Linkage |
|---|---|---|
| succinimidyl ester | primary amino, | carboxamide |
| | secondary amino | |
| sulpho-succinimidyl | primary amino, | carboxamide |
| ester | secondary amino | |
| isothiocyanate | amino groups | thiourea |
| haloacetamide | thiols, | thioether |
| maleimide | thiols | thioether |
| acid halide | amine | carboxamide |
| acid halide | hydroxyl | ester |
| acid hydrazide | carbonyl (aldehyde and | hydrazone |
| | ketone) | |
| dichlorotriazine | amine | amino triazinyl ether |
| dichlorotriazine | hydroxyl | triazinyl ether |
| phosphoramidite | hydroxyl | phosphate ester |

Particularly preferred reactive groups which are especially useful for labelling components with available amino and hydroxyl functional groups include:

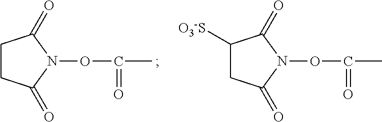

Particularly preferred reactive groups which are useful for labelling components with available thiol functional groups include:

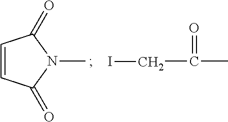

Particularly preferred examples of the group -L-R^{x} are those which comprise a carboxypentyl group, for example:

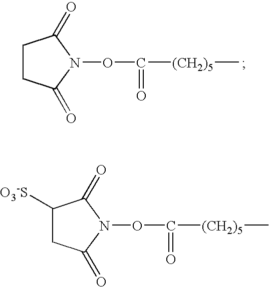

In another embodiment, R^{x} may be an affinity tag which is capable of binding specifically and non-covalently with its complementary specific binding partner. Examples of specific binding partner pairs include, but are not restricted to: biotin/avidin, biotin/streptavidin, polyhistidine tag-metal ion complexes with nitrilotriacetic acid (e.g. Ni²⁺: NTA). The complementary specific binding partner may be one component of a labelling complex for detection of a component. Thus, in one preferred labelling format, streptavidin, having four sites of attachment for a biotin label, may be used as a bridge linking a biotin group on the component with a dye according to the present invention wherein group R^{x} is biotin, iminobiotin or desthiobiotin. It is to be understood that in the context of the present invention, any two atoms or molecules that possess a specific binding affinity one for the other, may be employed. Preferred examples of affinity tags are selected from biotin, iminobiotin and desthiobiotin.

In preferred embodiment, one of the remaining R¹ or R² positions may be substituted by —(CH₂)ₖ—SO₃H, where k is hereinbefore defined. Preferably k is 3 or 4, i.e. the remaining R¹ or R² position may be substituted with either —(CH₂)₃— SO₃H or —(CH₂)₄—SO₃H. The use of cyanine dyes substituted directly or indirectly by fluorine and having additional substitution with one or more sulphonic acid groups for labelling biological molecules results in a labelled product in which there is reduced dye-dye aggregation and improved photostability, compared with cyanines having no such substitutions.

Alkyl is a straight or branched chain alkyl group containing from 1-4 carbon atoms, for example methyl, ethyl, n-propyl, iso-propyl and n-butyl and t-butyl.

Halide and halo groups are selected from chloride and chloro, bromide and bromo, and iodide and iodo.

Exemplary compounds of the according to the present invention are as follows:
i) 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate (Compound 1);
ii) 4-(2-{(1E,3E)-3-[1-(5-Carboxypentyl)-5,7-difluoro-3-methyl -3-(4-sulfobutyl)-1,3-dihydro-2H-indol-2-ylidene]prop-1-enyl}-6-fluoro-1,3-benzoxazol-3-ium-3-yl)butane-1-sulfonate (Compound 2);
iii) 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-5-fluoro-1,3-benzoxazol-3-ium-3-yl)butane-1-sulfonate (Compound 3);
iv) 4-[(2E)-2-((2E)-3-{3-[4-(Carboxymethyl)benzyl]-6-fluoro-1,3-benzoxazol-3-ium-2-yl}prop-2-enylidene)-4,5,6,7-tetrafluoro-3-methyl-3-(4-sulfobutyl)-2,3-dihydro-1H-indol-1-yl]butane-1-sulfonate (Compound 4);
v) 4-(2-{(1E,3Z)-3-[3-[4-(Carboxymethyl)benzyl]-6-fluoro-1,3-benzoxazol-2(3H)-ylidene]prop-1-enyl}-6-fluoro-1,3-benzoxazol-3-ium-3-yl)butane-1-sulfonate (Compound 5);
vi) 4-(2-{(1E,3Z)-3-[3-[4-(Carboxymethyl)benzyl]-6-fluoro-1,3-benzoxazol-2(3H)-ylidene]prop-1-enyl}-5,6-difluoro-1,3-benzoxazol-3-ium-3-yl)butane-1-sulfonate (Compound 6);
vii) 3-(5-Carboxypentyl)-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 7); and
viii) 3-{6-[(2,5-Dioxopyrrolidin-1-yl)oxy]-6-oxohexyl}-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 8).

The present invention also relates to fluorescently-labelled components and to labelling methods wherein the compounds of the present invention including at least one group -L-R^{x} attached to the R¹ and/or R² positions as hereinbefore defined may be used to label and thereby impart fluorescent properties to a component. In particular, the compounds of the present invention may be used for fluorescent labelling and detection of biological molecules, such as nucleic acids, DNA, RNA, oligonucleotides, nucleotides, proteins, peptides, antibodies, etc. Thus, in a second aspect, there is provided a method for labelling a component, the method comprising:
i) contacting said component with a compound of formula (I):

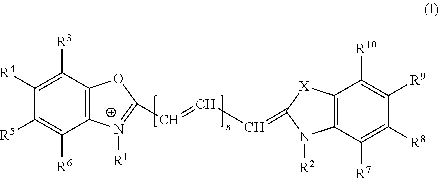

wherein:
X is selected from the group consisting of —O—, —S— and

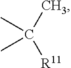

where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component; when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by -SO₃H or -(CF2)m-F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine; and
ii) incubating said compound with said component under conditions suitable for binding to and thereby labelling said component.

In one embodiment, X in the compound of formula (I) is the group:

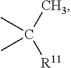

wherein R¹¹ is CH₃ or —(CH₂)ₖ—SO₃H;
at least one of groups R¹ and R² is the group -L-R^{x}, where L and R^{x} are hereinbefore defined;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

In another embodiment, X in the compound of formula (I) is —O—;
at least one of groups R¹ and R² is the group -L-R^{x}, where L and R^{x} are hereinbefore defined;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

In one preferred embodiment, at least one of groups R³, R⁴, R⁵, R⁶, R⁷, R⁸, R⁹ and R¹⁰ in the compounds of formula (I) and (II) is fluorine; more preferably, at least two of groups R³, R⁴, R⁵ and R⁶ and/or R⁷, R⁸, R⁹ and R¹⁰ are fluorine. Remaining groups R³, R⁴, R⁵, R⁶, R⁷, R⁸, R⁹ and R¹⁰ are selected from H or —SO₃H. Particularly preferred compounds are those in which each of the R³, R⁴, R⁵ and R⁶ positions and/or the R⁷, R⁸, R⁹ and R¹⁰ positions are substituted by fluorine.

In a second embodiment, the compounds of formula (I) may include a perfluoro C₁-C₄ alkyl substituent at one, preferably not more than two of the R³, R⁴, R⁵ or R⁶ positions and/or the R⁷, R⁸, R⁹ or R¹⁰ positions. Any remaining groups R³, R⁴, R⁵, R⁶, R⁷, R⁸, R⁹ and R¹⁰ are selected from H or F. Preferably, the perfluoro C₁-C₄ alkyl substituent is a trifluoromethyl substituent.

The group R^{x} is a group suitable for the formation of a covalent link between the compound of formula (I) and the component having a reactive or functional group as hereinbefore defined. The method comprises incubating the component to be labelled with an amount of the compound according to the invention under conditions such that the dye becomes covalently bound to the component. Methods for the formation of dye conjugates or complexes with components will be well known to the skilled person. For example, covalent labelling of proteins is typically performed in an aqueous buffered medium, suitably bicarbonate at pH 9.0, at ambient temperature for a period of typically 1 hour. The reaction is normally carried out in the dark. The labelled protein can be separated from any unreacted dye by size exclusion chromatography, for example using SEPHADEX™ as the stationary phase and phosphate buffer, pH 7.0 as the eluant. For multiple labelling of a biomolecule, the ratio of the amount or concentration of dye to the biomolecule should be adjusted accordingly.

In a third aspect there is provided a fluorescently-labelled dye conjugate of a component having the formula (I):

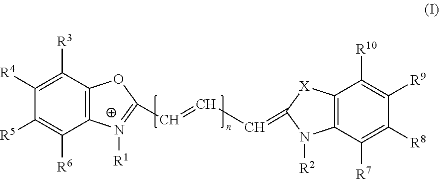

wherein:
X is selected from the group consisting of —O—, —S— and

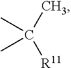

where R¹¹ is CH₃ or —(CH₂)ₖ—SO₃H;
at least one of groups R¹ and R² is the group -L-R^{p}, where L is a linking group having a chain from 1-20 linked atoms selected from the group consisting of carbon, nitrogen and oxygen atoms;
R^{p} is a component;
when either of groups R¹ and R² is not said group -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R³ or R⁹ taken in combination with R¹⁰ form a fused aromatic six-membered ring containing carbon atoms which may be optionally substituted one or more times by —SO₃H or —(CF2)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine.

Preferably, when X is —O— or —S—, groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4. More preferably, X is —O—.

Preferably, at least one of groups R³, R⁴, R⁵, R^{6,} R⁷, R⁸, R⁹ and R¹⁰ is fluorine.

Suitably, the dye conjugate includes a component that is selected from the group consisting of antibody, lipid, protein, peptide, carbohydrate, nucleotides which contain or are derivatized to contain one or more of an amino, sulphydryl, carbonyl, hydroxyl, carboxylic acid and thiophosphate groups, and oxy or deoxy polynucleic acids which contain or are derivatized to contain one or more of an amino, sulphydryl, carbonyl, hydroxyl, carboxylic acid and thiophosphate groups, microbial materials, drugs, hormones, cells, cell membranes and toxins.

In one embodiment, the dye of formula (I) is conjugated to a component comprising a biological targeting molecule. By the term “biological targeting moiety” (BTM) is meant a compound which, after administration, is taken up selectively or localises at a particular site of the mammalian body in vivo. Such sites may, for example, be implicated in a particular disease state, or be indicative of how an organ or metabolic process is functioning. The BTM may be of synthetic or natural origin, but is preferably synthetic. The term “synthetic” has its conventional meaning, i.e. man-made as opposed to being isolated from natural sources e.g. from the mammalian body. Such compounds have the advantage that their manufacture and impurity profile can be fully controlled. The BTM preferably comprises 3-100 mer peptides or peptide analogues which may be linear peptides or cyclic peptides or combinations thereof; or enzyme substrates, enzyme antagonists or enzyme inhibitors; synthetic receptor-binding compounds; oligonucleotides, or oligo-DNA or oligo-RNA fragments. When the BTM is a peptide, it is preferably a 4-30 mer peptide, and most preferably a 5-28 mer peptide.

In a fourth aspect, there is provided a pharmaceutical composition which comprises the conjugate of the third aspect, together with a biocompatible carrier, in a form suitable for mammalian administration. Preferably, the fluorescent dye is conjugated to a component comprising a BTM as defined hereinbefore.

Suitably, the “biocompatible carrier” is a fluid, especially a liquid, in which the conjugate can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethylene glycols, propylene glycols and the like). Preferably the biocompatible carrier is pyrogen-free water for injection or isotonic saline.

The fluorescent dye-labelled component according to the present invention may subsequently be used as a reagent for analysis or detection, for example in microwell plates, gels and in cell based assays. By the term “optical imaging” is meant any method that forms an image for detection, for example, by means of a charge coupled device (CCD) imager (such as a scanning imager or an area imager). The LEADSEEKER™ system features a CCD camera allowing fluorescence imaging of assays performed in high density microwell plates in a single pass. Imaging is quantitative and fast, and instrumentation suitable for imaging applications can now simultaneously image the whole of a multiwell plate.

Alternatively, the fluorescent dye-labelled conjugate of a component such as a BTM may be administered in vivo to a suitable animal model. Thus, in one embodiment, there is provided a method of in vivo optical imaging of the mammalian body which comprises use of either the dye-conjugate of a BTM or pharmaceutical composition thereof in order to obtain images of sites of BTM localisation in vivo, based on interaction with light in the green to near-infrared region (wavelength 500-1200 nm). Optical imaging further includes all methods from direct visualization without use of any device and involving use of devices such as various scopes, catheters and optical imaging equipment, e.g. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (Editor): “Biomedical Photonics Handbook” (2003), CRC Press LCC; Mycek & Pogue (Editors): “Handbook of Biomedical Fluorescence” (2003), Marcel Dekker, Inc.; Splinter & Hopper: “An Introduction to Biomedical Optics” (2007), CRC Press LCC.

In addition to the foregoing one-step labelling process, the present invention also relates to two-step labelling and detection processes in which, in a first step, a compound according to the present invention including at least one group -L-R^{x} attached to the R¹ and/or R² positions as hereinbefore defined may be used to label and thereby impart fluorescent properties to a primary component, such as an antibody, protein, DNA probe, etc. In the second step of the process, the fluorescently labelled primary component is then used as a probe for detection of a secondary component, such as an antigen for which the antibody is specific. Thus, in a fifth aspect, there is provided a method for detecting a secondary component in a sample comprising the steps of:
i) contacting a sample containing or suspected to contain the secondary component to be detected with a primary component under conditions to form a complementary specific binding pair and wherein said primary component is labelled with a compound of formula (I):

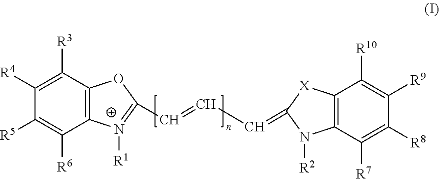

wherein:
X is selected from the group consisting of —O—, —S— and

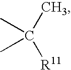

where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component; when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine;

ii) binding said labelled primary component to said second component to form a labelled secondary component; and
iii) detecting said labelled secondary component by an optical method.

In one embodiment, X in the compound of formula (I) is the group:

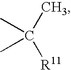

wherein R¹¹ is CH₃ or —(CH₂)ₖ—SO₃H;
at least one of groups R¹ and R² is the group -L-R^{x}, where L and R^{x} are hereinbefore defined;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

In another embodiment, X in the compound of formula (I) is —O—; at least one of groups R¹ and R²is the group -L-R^{x}, where L and R^{x} are hereinbefore defined;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

The two step labelling and detection method of the present invention can be applied to any molecules which possess a specific binding affinity for each other. Thus, the dyes of the present invention may be used for labelling one component of a complementary specific binding pair, which labelled component may in turn be used in the detection of binding to the other component of the complementary specific binding pair. Examples of complementary specific binding pairs include, but are not restricted to, antibody/antigen, lectin/glycoprotein, biotin/avidin, biotin/streptavidin, hormone/receptor, enzyme/substrate or co-factor, DNA/DNA, DNA/RNA and DNA/binding protein.

In one application, the dyes of the present invention may be used in Western Blotting applications, where they may be employed in conjunction with fluorescent dye labels (CY™3 λₘₐₓ 570 nm and CY™5, λₘₐₓ 670 nm), thereby enabling three-colour, multiwavelength fluorescent detection. In an example of this technique, multiplex protein detection is possible using ECL Plex fluorescent Western Blotting System (GE Healthcare). According to this method, proteins may be detected following electrophoretic separation, for example by means of a polyacrylamide gel, by blotting the gel onto a low fluorescence nitrocellulose membrane. The blots are then incubated with protein specific antibodies, followed by detection using fluorescent dye-labelled species specific secondary antibodies. See for example, Ausubel, et al, (Eds), (1994), Current Protocols in Molecular Biology, Vol. 1, John Wiley & Sons, Inc., 10.8.1-10.8.16. In another example, an appropriately reactive fluorescent compound of the invention may be conjugated to a DNA or RNA fragment and the resultant fluorescently-labelled conjugate then caused to bind to a complementary strand of DNA or RNA. The dye-labelled components may be detected and/or quantitated by optical means, suitably fluorescence microscopy employing an imaging instrument, such as a CCD camera, fluorescence scanner or confocal imager.

The present invention relates to intermediates and to methods suitable for preparing the dyes of formula (I). Thus, in a sixth aspect, there is provided a compound of formula (A):

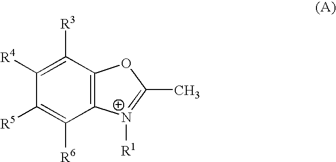

wherein:
R¹ is selected from —(CH₂)ₖ—SO₃H, -L-R^{x} and -L-R^{p} where L is a linking group having a chain from 1-20 linked atoms selected from the group consisting of carbon, nitrogen and oxygen atoms;
R^{x} is a group suitable for covalent attachment of said compound to a component;
R^{p} is a component;
groups R³, R⁴, R⁵ and R⁶ are selected independently from hydrogen, —SO₃H and the group —(CF₂)ₘ—F, where m is 0 or an integer from 1 to 4;
provided that at least one of groups R³, R⁴, R⁵ and R⁶ comprises fluorine.

Preferably, at least one of groups R³, R⁴, R⁵ and R⁶ is fluorine. More preferably, at least two of groups R³, R⁴, R⁵ and R⁶ are fluorine. Remaining groups R³, R⁴, R⁵ and R⁶ are selected from H or —SO₃H. Particularly preferred are compounds of formula (A) in which each of the R³, R⁴, R⁵ and R⁶ positions is substituted by fluorine.

Alternatively, compounds of formula (A) may include a perfluoro C₁-C₄ alkyl substituent at one, preferably not more than two of the R³, R⁴, R⁵ or R⁶ positions. Any remaining groups R³, R⁴, R⁵ and R⁶ are selected from H or F. Preferably, the perfluoro C₁-C₄ alkyl substituent is a trifluoromethyl substituent.

In a seventh aspect, compounds according to the invention may be prepared by a process comprising:
a) reacting a first compound having the formula (A):

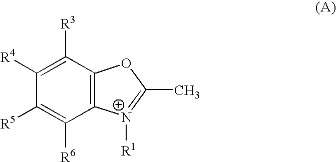

with
b) a second compound which may be the same or different from the first compound and having the formula (B):

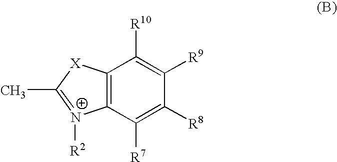

and
c) a third compound (C) suitable for forming a conjugated linkage between said first and second compounds;
wherein:
X is selected from the group consisting of —O—, —S— and

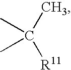

where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component;
when either of groups R¹ and R² is not said group -L-R^{x}, said remaining group R¹ or R² is selected from C₁-C₄ alkyl and —(CH₂)ₖ—SO₃H; groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined; and
k is an integer from 1 to 10;
provided that at least one of groups R³, R⁴, R⁵, R⁶, R⁷, R⁸, R⁹ and R¹⁰ comprises fluorine.

Preferably, when X is —O— or —S—, groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4.

Preferably, —(CH₂)ₖ—SO₃H is selected from —(CH₂)₃—SO₃H and —(CH₂)₄—SO₃H.

According to the method, intermediate compounds (A), (C) and (B) may be reacted either in a single step or in a multiple step process to form the compounds of formula (I). Symmetrical compounds of formula (I) wherein structures (A) and (B) are the same may be suitably prepared by reacting a compound of formula (A) (or (B)) in two molar proportions with an appropriate bis-functional methine fragment containing 1, 3 or 5 carbon atoms. For example, a substituted N,N′-diphenylformamidine, or an ortho ester will be employed as the third compound (C) for preparing trimethine cyanine dye analogues. In a corresponding manner, a suitably substituted malondialdehyde dianil may be employed for preparing the pentamethine cyanine dye analogues and a glutaconic aldehyde for preparing heptamethine cyanine dye analogues. The reaction is usually carried out in an organic solvent, such as pyridine and heated to reflux. The mixture subsequently is cooled and poured into an organic solvent such as ether. The resulting solid or semi-solid may be purified by chromatography on a silica gel column using a series of methanol/chloroform solvents.

Unsymmetrical compounds of formula (I) wherein structures (A) and (B) are different may be conveniently prepared in a two step process. In this process, an intermediate compound is first formed by reacting an indolium compound of formula (A) with a compound suitable for forming the linkage, for example, a suitably substituted N,N′-diphenylformamidine, or malonaldehyde dianil, in the presence of acetic anhydride, to form a 2-anilinovinyl or 4-anilino-1,3-butadienyl quaternary salt. The intermediate quaternary salt may be reacted with a second 2-methyl indolium quaternary salt to give a compound of formula (I). Alternative intermediates for forming the polymethine linkage joining the heterocyclic ring systems are known and are described, for example in Hamer, F. M., “The Cyanine Dyes and Related Compounds”, Interscience (1964).

CY™ is a trademark of GE Healthcare UK Limited.

### BRIEF DESCRIPTION OF THE DRAWINGS

FIG. 1 is a comparison of the photostability of anti-mouse IgG conjugated to Compound 8 and to a non-fluorinated cyanine dye, CY™2.

FIG. 2 is a scan showing the detection of anti-actin IgG by goat anti-mouse IgG labelled with Compound 8.

### EXAMPLES

The present invention will be described in more detail by way of examples, which however are in no way intended to limit the scope of the present invention as defined by the appended claims. All references given below or elsewhere in the present specification are hereby included herein by reference.

### General Procedure for Preparation of Fluorinated 2-Methylbenzoxazoles

A general method for the preparation of benzoxazole derivatives from substituted 2-aminophenols and trialkyl orthoesters is described by Iraj Mohammadpoor-Baltork, Ahmad R. Khosropour, and Seyedeh F. Hojati, Monatshefte für Chemie (2007), 138, 663-667. The following examples demonstrate the application of the above method to the preparation of fluorinated 2-methylbenzoxazoles.

### 1. 6-Fluoro-2-methylbenzoxazole

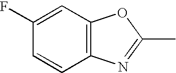

2-Amino-5-fluorophenol (950 mg, 7.5 mmol, 1.0 eq), triethyl orthoacetate (1.51 ml, 8.25 mmol, 1.1 eq) and bismuth (III) trifluoromethanesulfonate (50 mg, 0.075 mmol, 0.01 eq) were mixed and stirred at ambient temperature for 30 mins. The resulting solution was then diluted with dichloromethane and purified by silica flash chromatography (0-2% methanol/dichloromethane) to give the title compound as a clear liquid. 725 mg. The liquid transformed to a crystalline solid at temperatures below 5° C. This product analyzed identically to an authentic sample from a commercial source (Aldrich Chemical Company).

### 2. 5-Fluoro-2-methylbenzoxazole

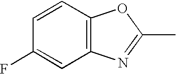

2-Amino-4-fluorophenol (1.01 g, 7.95 mmol, 1.0 eq), triethyl orthoacetate (1.60 ml, 8.7 mmol, 1.1 eq) and bismuth (III) trifluoromethanesulfonate (50 mg) were mixed and stirred at ambient temperature for 30 mins. The resulting solution was then diluted with dichloromethane and purified by silica flash chromatography (dichloromethane) to give the title compound as a clear liquid, 1.045 g (87%). The liquid transformed to a crystalline solid at temperatures below 5° C. MS (MALDI-TOF): MH⁺=152. UV/VIS (MeOH): ABS λₘₐₓ=282, 276 & 230 nm. δH/ppm (400 MHz, CDCl₃): 2.60 (s, 3H), 7.02 (1H, td), 7.33 (1H, dd) and 7.39 (1H, dd).

### 3. 5,6-Difluoro-2-methylbenzoxazole

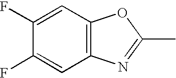

2-Amino-4,5-difluorophenol (1.00 g, 76.9 mmol, 1.0 eq), triethyl orthoacetate (1.40 ml, 7.6 mmol, 1.1 eq) and bismuth (III) trifluoromethanesulfonate (50 mg) were mixed and stirred at ambient temperature for 30 mins. The resulting solution was then diluted with dichloromethane and purified by silica flash chromatography (dichloromethane) to give the title compound as a white crystalline solid, 1.035 g (89%). MS (MALDI-TOF): MH⁺=170. UV/VIS (MeOH): ABS λₘₐₓ=282, 278, 273 & 227 nm. δH/ppm (400 MHz, CDCl₃): 2.65 (s, 3H), 7.33 (1H, dd) and 7.44 (1H, dd).

4. 5,7-Difluoro-2-methylbenzoxazole

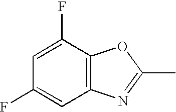

2-Amino-4,6-difluorophenol (1.00 g, 76.9 mmol, 1.0 eq), triethyl orthoacetate (1.40 ml, 7.6 mmol, 1.1 eq) and bismuth (III) trifluoromethanesulfonate (50 mg) were mixed and stirred at ambient temperature for 30 mins. The resulting solution was then diluted with dichloromethane and purified by silica flash chromatography (dichloromethane) to give the title compound as a crystalline solid, 1.088 g (93%). MS (MALDI-TOF): MH⁺=170. UV/VIS (MeOH): ABS λₘₐₓ=278, 268 & 231 nm. δH/ppm (400 MHz, CDCl₃): 2.66 (s, 3H), 6.85 (1H, td) and 7.16 (1H, ddd).

### General Method for N-alkylation of 5-fluoro and 6-fluoro-2-methyl-benzoxazole by Formation of N-carboxyalkyl and N-sulfoalkyl Derivatives

N-Alkylation of 5(6)-fluoro-2-methylbenzoxazole to form N-carboxyalkyl- and N-sulfoalkyl derivatives may be performed by methods analogous to those described elsewhere for indolenine analogues (see for example Mujumdar, R. B. et al, Bioconjugate Chemistry, (1993), 4, 105-111).

### 5. 3-[4-(Carboxymethyl)benzyl]-6-fluoro-2-methyl-1,3-benzoxazol-3-ium bromide

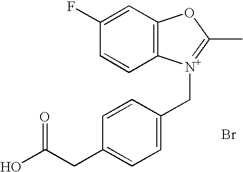

6-Fluoro-2-methylbenzoxazole (from Example 1, 700 mg, 4.6 mmol) and 4-(bromomethyl)phenylacetic acid (700 mg, 3.1 mmol) were mixed and heated to effect reaction (oil bath, 140° C. for 30 mins, 120° C. for 16 hrs). The solids initially dissolved to give a slightly cloudy liquid, which solidified upon overnight reaction. The solid mass was allowed to cool to room temperature and triturated with diethyl ether to a fine slurry, from which the solids were isolated by centrifugation and decantation. The solid pellet was resuspended in ethyl acetate, centrifuged and the liquors decanted; the operation was then repeated with ether before vacuum drying the result. The crude product salt was then used for dye syntheses without further purification.

6. 4-(6-Fluoro-2-methyl-1 3-benzoxazol-3-ium-3-yl)butane-1-sulfonate

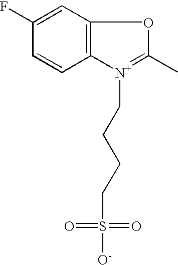

6-Fluoro-2-methylbenzoxazole (Aldrich Catalogue Code No.538434, 500 mg, 3.3 mmol) and 1,4-butanesultone (2.50 ml) were mixed and heated under nitrogen at 110°C. for 16 hrs. The reaction mix was then allowed to cool to room temperature and triturated with diethyl ether to give an immiscible gum. The liquors were decanted, the gum washed with more ether and dried under vacuum. The crude product salt was then used for dye syntheses without further purification.

### 7. 4-(5-Fluoro-2-methyl-1 3-benzoxazol-3-ium-3-yl)butane-1-sulfonate

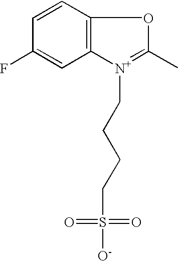

5-Fluoro-2-methylbenzoxazole (from Example 2, 500 mg, 3.3 mmol) and 1,4-butanesultone (2.50 ml) were mixed and heated under nitrogen at 110° C. for 16 hrs. The reaction mix was then allowed to cool to room temperature and triturated with diethyl ether to give an immiscible gum. The liquors were decanted, the gum washed with more ether and dried under vacuum. The crude product salt was then used for dye syntheses without further purification.

8. 4-(5,6-Difluoro-2-methyl-1,3-benzoxazol-3-ium-3-yl)butane-1-sulfonate

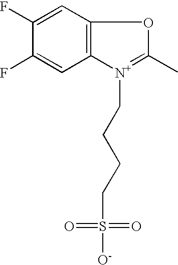

5,6-Difluoro-2-methylbenzoxazole (from Example 3, 500 mg, 3.3 mmol) and 1,4-butanesultone (2.50 ml) were mixed and heated under nitrogen at 110° C. for 16 hrs. The reaction mix was then allowed to cool to room temperature and triturated with diethyl ether to give an immiscible gum. The liquors were decanted, the gum washed with more ether and dried under vacuum. The crude product salt was then used for dye syntheses without further purification.

### 9. 4-(5,7-Difluoro-2-methyl-1,3-benzoxazol-3-ium-3-yl)butane-1-sulfonate

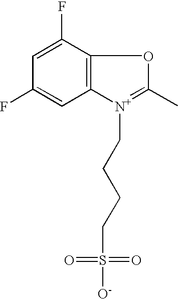

5,7-Difluoro-2-methylbenzoxazole (from Example 4, 100 mg, 0.6 mmol) and 1,4-butanesultone (0.50 ml) were mixed and heated under nitrogen at 110° C. for 16 hrs. The reaction mix was then allowed to cool to room temperature and triturated with diethyl ether to give a small quantity of immiscible gum. The liquors were decanted, the gum washed with more ether and dried under vacuum. The crude product salt was then used for dye syntheses without further purification.

### 10. 4-[1-(5-Carboxypentyl)-4,5,6,7-tetrafluoro-2,3-dimethyl-3H-indolium-3-yl]butane-1-sulfonate

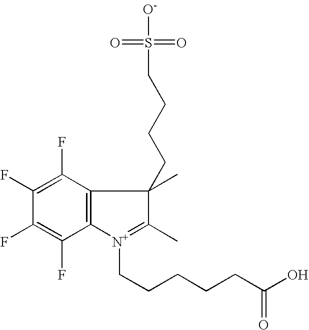

### 10.1 5-(Ethoxycarbonyl)-5-methyl-6-oxoheptane-1-sulphonate, sodium salt

Sodium hydride (60 wt %, 12 g≡0.3 mol NaH) was slurriedin dry DMF (100 ml). The resulting suspension was cooled with stirring to 0° C. To this was added a solution of ethyl 2-methylacetoacetate (50 g, 0.346 mol) in DMF (25 ml), dropwise so as to maintain the temperature at <10° C. and control effervescence. Once addition was complete and hydrogen evolution ceased, the mixture was warmed in a warm water bath until a clear, pale yellow solution resulted. This was cooled again to 0° C. A solution of 1,4-butanesultone (45 g, 0.33 mol) in DMF (25 ml) was added over 15 mins, maintaining the temperature at <10° C. Once addition was complete, the mixture was heated at 50° C. for 16 hrs. The solvent was then evaporated under vacuum to dryness; the residue was partitioned between water and diethyl ether. The aqueous layer was retained; the organic layer was extracted with fresh water, then discarded. The combined aqueous extracts were washed with fresh ether, then evaporated under vacuum to give the product as a waxy solid. δH/ppm (270 MHz; D₂O) 4.23 (2H, q), 2.9 (2H, app t), 2.26 (3H, s), 2.0-1.6 (6H, m), 1.36 (3H, s) and 1.26 (3H, t).

### 10.2 5-Methyl-6-oxoheptane-1-sulphonic acid

5-(Ethoxycarbonyl)-5-methyl-6-oxoheptane-1-sulphonate, sodium salt (from Example 10.1) was heated at 90° C. in concentrated hydrochloric acid (200 ml), until TLC indicated complete reaction (˜3 hrs). The solvent was then evaporated under vacuum; the residue was purified by flash chromatography (Silica. Ethanol/dichloromethane mixtures) to give 49.6 g of 5-methyl-6-oxoheptane-1-sulphonic acid. δH/ppm (270 MHz; D₂O) 2.9 (2H, app t), 2.68 (1H, m), 2.2 (3H, s), 1.8-1.3 (6H, m) and 1.18 (3H, d).

### 10.3 2,3-Dimethyl-3-(4-sulfobutyl)-4,5,6,7-tetrafluoro-3H-indole, disodium salt

2,3,4,5-Tetrafluoroaniline (1.75 g, 0.01M) was dissolved in conc. HCl (280 ml). The flask was maintained at −10° C. and a solution of NaNO₂ (1 eq) in water (10 ml) added dropwise followed subsequently by a solution of tin(II) chloride (3.4 g) in conc. HCl (40 ml). The reaction was returned to ambient temperature and stirred for 1 hour. The solvent was removed in vacuo to yield the crude product as a yellow salt (7 g).

The hydrazine salts and crude material were dissolved in acetic acid (50 ml) with the sulfonated ketone, 5-methyl-6-oxoheptane-1-sulphonic acid (6 g). The solution was heated at 140° C. for 2 hrs to yield an orange solution with fine orange precipitate. The solvent was evaporated to yield a brown gum. The product was isolated by reverse phase HPLC (0.1% TFA, water/acetonitrile gradient) to yield the product. MS (MALDI-TOF): MH⁺=354.

### 10.4 4-[1-(5-Carboxypentyl)-4,5,6,7-tetrafluoro-2,3-dimethyl-3H-indolium-3-yl]butane-1-sulfonate

Tetra-fluorinated indole (from 10.3) (150 mg, 4.2×10⁻⁴ mol, 1 eq.) was heated at 140° C. with bromo-hexanoic acid (15 g, 0.073 mol, 260 eq) for 24 hr under nitrogen. The product was triturated with diethyl ether and dried under vacuum to yield a brown mass. The major constituent was confirmed as 4-[1-(5-carboxypentyl)-4,5,6,7-tetrafluoro-2,3-dimethyl-3H-indolium-3-yl]butane-1-sulfonate by LC-MS and was used without further purification. MS (MALDI-TOF): MH⁺=470.

### 11. 4-[4,5,6,7-Tetrafluoro-2,3-dimethyl-3-(4-sulfobutyl)-3H-indolium-1-yl]butane-1-sulfonate

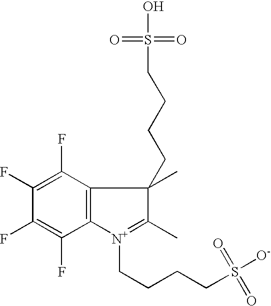

Tetra-fluorinatedindole (from Example 10.3, 100 mg, 2.8×10⁻⁴ mol, 1 eq.) was heated at 140° C. with butane sultone (10 g, 0.073 mol) for 24 hr under nitrogen. The product was triturated with diethyl ether and dried under vacuum to yield a brown mass. The major constituent was confirmed as 4-[4,5,6,7-tetrafluoro-2,3-dimethyl-3-(4-sulfobutyl)-3H-indolium-1-yl]butane-1-sulfonate by LC-MS and was used without further purification. MS (MALDI-TOF): MH⁺=490.

### 12. 4-[1-(5-Carboxypentyl)-5,7-difluoro-2,3-dimethyl-3H-indolium-3-yl]butane-1-sulfonate

### 12.1 5,7-Difluoro-2,3-dimethyl-3-(4-sulfobutyl)-3H-indole

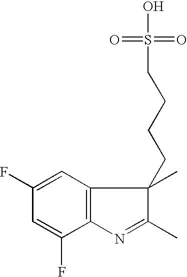

To 2,4-difluorophenyl hydrazine hydrochloride (2 g) in acetic acid (60 ml) was added 5-methyl-6-oxoheptane-1-sulphonic acid (4.5 g) and the solution heated to reflux for 2 hrs. The volatiles were removed on a rotary evaporator to give the crude product, which was purified by flash chromatography (RP-18 silica, water/acetonitrile mixtures as eluant). The relevant fractions (identified by LC-MS) were combined, concentrated on a rotary evaporator and freeze-dried to give the desired product (6 g). MS (MALDI-TOF), MH⁺=317.

### 12.2 4-[1-(5-Carboxypentyl)-5,7-difluoro-2,3-dimethyl-3H-indolium-3-yl]butane-1-sulfonate

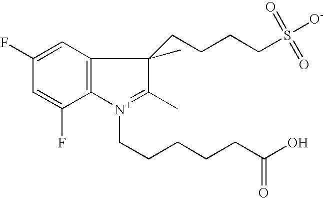

To 5,7-difluoro-2,3-dimethyl-3-(4-sulfobutyl)-3H-indole (1.0 g) was added 6-bromohexanoic acid (5 g) and the solution heated to 140° C. for 2 days. On cooling, the product was triturated with diethyl ether to give a slurry. The solids were collected by filtration, washed with ether and dried under vacuum to give the crude product. This was further purified by preparative HPLC to give the title product (300 mg). MS (MALDI-TOF), MH⁺=432.

### 13. 3-(5-Carboxypentyl)-2-methyl-1,3-benzoxazol-3-ium-6-sulfonate

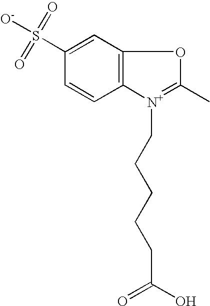

### 13.1 2-Methyl-6-sulfobenzoxazole

Concentrated sulfuric acid (37.5 g) was cooled in an ice bath to 4° C. 2-methylbenzoxazole (Aldrich, Code No. 27,097-0) (22.5 ml, 25 g) was added slowly dropwise with the formation of white crystals. 20% Oleum (Aldrich, Code No. 32,355-1) (37.5 ml) was added slowly dropwise to the chilled mixture, followed by ferric chloride (125 mg). The reaction mixture was heated to 125° C. in an oil bath for 1.75 hours. The reaction mixture was then added dropwise to chilled (4° C.) acetone and the mixture allowed to stir for 15 minutes at 4° C. The brown/purple solid was collected by filtration, washed with acetone (2×100 ml) and dried. The product, 2-methyl-6-sulfobenzoxazole was converted to its potassium salt as a light brown solid by neutralisation with KOH in 2-propanol.

### 13.2 3-(5-Carboxypentyl)-2-methyl-1,3-benzoxazol-3-ium-6-sulfonate

To 2-methyl-6-sulfobenzoxazole (5 g) in warmed (30° C.) tetramethylene sulfone (25 g) was added 6-bromohexanoic acid (15 g) in three equal portions. The reaction mixture was heated to 140° C. overnight, then cooled to approx. 40° C. and then added to ethyl acetate (200 ml). The product, 2-methyl-3-carboxypentyl-6-sulfobenzoxazole, was collected by filtration, washed with ethyl acetate (2×50 ml) and dried under vacuum.

### Dye Synthesis

### 14. 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate (Compound 1)

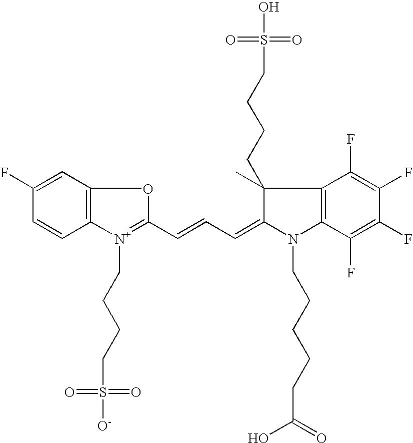

### 14.1 4-{2-[(E)-2-Anilinovinyl]-6-fluoro-1,3-benzoxazol-3-ium-3-yl}butane-1-sulfonate

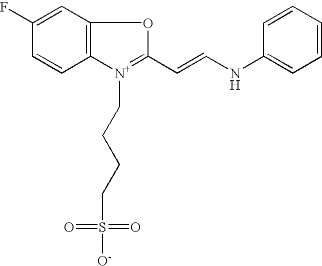

To 4-(6-fluoro-2-methyl-1,3-benzoxazol-3-ium-3-yl)butane-1-sulfonate (example 6, ˜3 mmol crude material) were added N,N′-diphenylformamidine (500 mg), triethyl orthoformate (1.65 ml) and ethanol (5 ml). The mixture was heated at 100° C. for 2.5 hrs, then cooled and the solvent evaporated under vacuum. The residue was triturated with diethyl ether, then ethyl acetate before purification by flash chromatography (silica, methanol/dichloromethane). The title compound was isolated as a pale yellow solid, 413 mg. MS (MALDI-TOF): MH⁺=391. UV/VIS (MeOH): ABS λₘₐₓ=385 nm.

### 14.2 4-(2-{(1E,3E)-3-[1-(5-Carboxypentyl)-4 5,6 7-tetrafluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate

4-{2-[(E)-2-Anilinovinyl]-6-fluoro-1,3-benzoxazol-3-ium-3-yl}butane-1-sulfonate (from Example 14.1, 60 mg) and 4-[1-(5-carboxypentyl)-4,5,6,7-tetrafluoro-2,3-dimethyl-3H-indolium-3-yl]butane-1-sulfonate (from Example 10, 50 mg) were dissolved in pyridine (900 μl) and acetic acid (900 μl), then acetic anhydride (200 μl) added. The mixture was heated at 100° C. for 3 hrs, then cooled and the solvent evaporated under vacuum. The residue was triturated with diethyl ether, then purified by HPLC (C₁₈, water+0.1% TFA vs acetonitrile). Appropriate fractions were pooled, evaporated under vacuum to low volume and freeze-dried to give the product dye. MS (MALDI-TOF): MH⁺=765. UV/VIS (MeOH): ABS λₘₐₓ=504 nm. Fluorescence (MeOH): excitation λₘₐₓ=503 nm; emission λₘₐₓ=529 nm. Accurate mass: MH⁺=C₃₃H₃₈N₂F₅O₉S₂⁺ requires 765.1939, found MH⁺=765.1981 (5.5 ppm).

### 15. 4-(2-{(1E,3E)-3-[1-(5-Carboxypentyl)-5,7-difluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate (Compound 2)

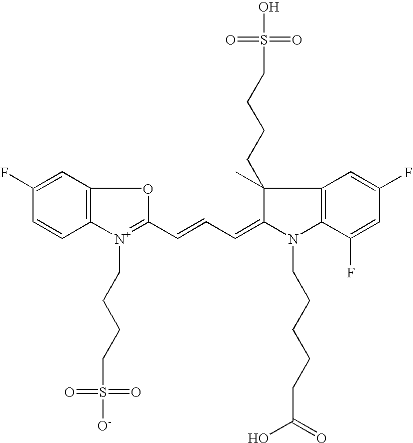

4-{2-[(E)-2-Anilinovinyl]-6-fluoro-1,3-benzoxazol-3-ium-3-yl}butane-1-sulfonate (from Example 14.1, 60 mg) and 4-[1-(5-carboxypentyl)-5,7-difluoro-2,3-dimethyl-3H-indolium-3-yl]butane-1-sulfonate (from example 12, 45 mg) were dissolved in pyridine (900 μl) and acetic acid (900 μl), then acetic anhydride (200 μl) added. The mixture was heated at 100° C. for 3 hrs, then cooled and the solvent evaporated under vacuum. The residue was triturated with diethyl ether, then purified by HPLC (C₁₈, water+0.1% TFA vs acetonitrile). Appropriate fractions were pooled, evaporated under vacuum to low volume and freeze-dried to give the product dye. MS (MALDI-TOF): MH⁺=729. UV/VIS (MeOH): ABS λₘₐₓ=511 nm. Fluorescence (MeOH): excitation λₘₐₓ=511 nm; emission λₘₐₓ=535 nm.

### 16. 4-(2-{(1E,3E)-3-[1-(5-carboxypentyl)-4,5,6,7-tetrafluoro-3-methyl-3-(4-sulfobutyl)-1,3-dihydro-2H-indol-2-ylidene]prop-1-enyl}-5-fluoro-1,3-benzoxazol-3-ium-3-yl)butane-1-sulfonate (Compound 3)

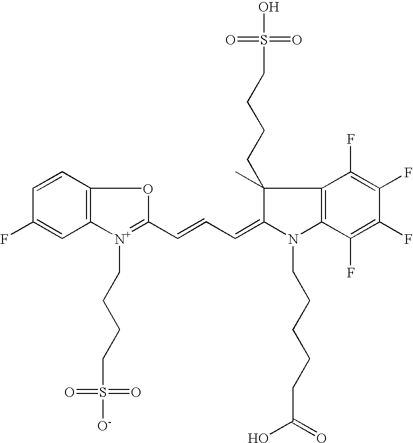

### 16.1 4-{2-[(E)-2-Anilinovinyl]-5-fluoro-1,3-benzoxazol-3-ium-3-yl}butane-1-sulfonate

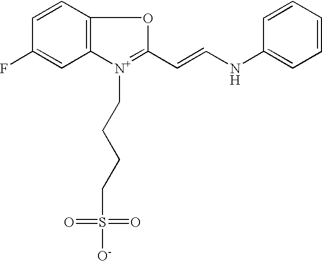

To a flask containing 4-(5-fluoro-2-methyl-1,3-benzoxazol-3-ium-3-yl)butane-1-sulfonate (from Example 7, ˜3 mmol of crude material) were added N,N′-diphenylformamidine (650 mg), triethyl orthoformate (0.55 ml) and ethanol (5 ml). The mixture was heated at 100° C. for 3 hrs, during which time the initial solution deposited a pale yellow precipitate. The mixture was then cooled and the solid collected by vacuum filtration, washed with ethyl acetate and dried under vacuum at 40° C. The title compound was isolated as a pale yellow solid, 305 mg. MS (MALDI-TOF): MH⁺=391. UV/VIS (MeOH): ABS λₘₐₓ =386 nm.

### 16.2 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-5-fluoro-1,3-benzoxazol-3-ium-3-yl)butane-1-sulfonate

4-{2-[(E)-2-Anilinovinyl]-5-fluoro-1,3-benzoxazol-3-ium-3-yl}butane-1-sulfonate (from Example 16.1, 50 mg) and 4-[1-(5-carboxypentyl)-4,5,6,7-tetrafluoro-2,3-dimethyl-3H-indolium-3-yl]butane-1-sulfonate (from Example 10, 50 mg) were dissolved in pyridine (900 μl) and acetic acid (900 μl), then acetic anhydride (200 μl) added. The mixture was heated at 100° C. for 3 hrs, then cooled and the solvent evaporated under vacuum. The residue was triturated with diethyl ether, then purified by HPLC (C₁₈, water+0.1% TFA vs acetonitrile). Appropriate fractions were pooled, evaporated under vacuum to low volume and freeze-dried to give the product dye. MS (MALDI-TOF): MH⁺=765. UV/VIS (MeOH): ABS λₘₐₓ=506 nm. Fluorescence (MeOH): excitation λₘₐₓ =508 nm; emission λₘₐₓ=531 nm.

### 17. 4-[(2E)-2-((2E)-3-{3-[4-(carboxymethyl)benzyl]-6-fluoro-1,3-benzoxazol-3-ium-2-yl}prop-2-enylidene)-4,5,6,7-tetrafluoro-3-methyl-3-(4-sulfobutyl)-2,3-dihydro-1H-indol-1-yl]butane-1-sulfonate (Compound 4)

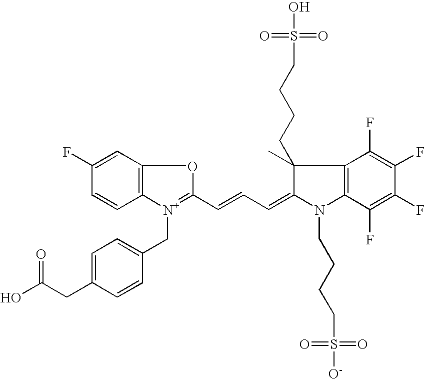

### 17.1 2-[(E)-2-anilinovinyl]-3-[4-(carboxymethyl)benzyl]-6-fluoro-1,3-benzoxazol-3-ium acetate

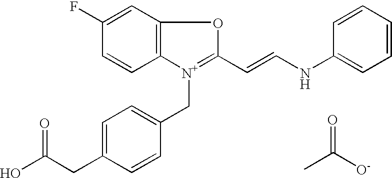

To a flask containing 3-[4-(carboxymethyl)benzyl]-6-fluoro-2-methyl-1,3-benzoxazol-3-ium bromide (from example 5, 300 mg crude solid) were added N,N′-diphenylformamidine (200 mg) and acetic acid (1.5 ml). The mixture was heated at 100° C. for 5 hrs, giving a yellowish solution. The mixture was then cooled and the solvent evaporated under vacuum. The residue was triturated with diethyl ether and the solids collected, washed with ethyl acetate and air-dried. It was then purified by HPLC (C₁₈, water+0.1 % AcOH vs acetonitrile). Appropriate fractions were pooled, evaporated under vacuum to low volume and freeze-dried to give the product dye as a light yellow solid, 71 mg. MS (MALDI-TOF): MH⁺=403. UV/VIS (MeOH): ABS λₘₐₓ=385 nm.

### 17.2 4-[(2E)-2-((2E)-3-{3-[4-(carboxymethyl)benzyl]-6-fluoro-1,3-benzoxazol-3-ium-2-yl{prop-2-enylidene)-4,5,6,7-tetrafluoro-3-methyl-3-(4-sulfobutyl)-2,3-dihydro-1H-indol-1-yl]butane-1-sulfonate

2-[(E)-2-Anilinovinyl]-3-[4-(carboxymethyl)benzyl]-6-fluoro-1,3-benzoxazol-3-ium acetate (from Example 17.1, 30 mg) and 4-[4,5,6,7-tetrafluoro-2,3-dimethyl-3-(4-sulfobutyl)-3H-indolium-1-yl]butane-1-sulfonate (from Example 11, 60 mg) were dissolved in pyridine (900 μl) and acetic acid (900 μl), then acetic anhydride (200 μl) added. The mixture was heated at 100° C. for 3 hrs, then cooled and the solvent evaporated under vacuum. The residue was triturated with diethyl ether, then purified by HPLC (C₁₈, water+0.1% TFA vs acetonitrile). Appropriate fractions were pooled, evaporated under vacuum to low volume and freeze-dried to give the product dye. MS (MALDI-TOF): MH⁺=799. UV/VIS (MeOH): ABS λₘₐₓ=508 nm. Fluorescence (MeOH): excitation λₘₐₓ=509 nm; emission λₘₐₓ=532 nm.

### 18. 4-(2-{(1E,3Z)-3-[3-[4-(Carboxymethyl)benzyl]-6-fluoro-1,3-benzoxazol-2(3H)-ylidene]prop-1-enyl}-6-fluoro-1,3-benzoxazol-3-ium-3-yl)butane-1-sulfonate (Compound 5)

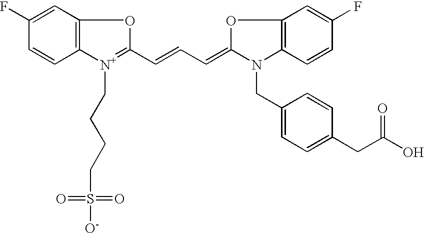

4-(6-Fluoro-2-methyl-1,3-benzoxazol-3-ium-3-yl)butane-1-sulfonate (from Example 6, ˜50 mg) and 3-[4-(carboxymethyl)benzyl]-6-fluoro-2-methyl-1,3-benzoxazol-3-ium bromide (from Example 5, 50 mg) were dissolved in pyridine (1.5 ml), with warming and sonication. Triethyl orthoformate (0.5 ml) was then added and the resulting mixture heated at 120° C. for 3 hrs to give a deep yellow solution. This was cooled and the solvent evaporated under vacuum. The residue was triturated with diethyl ether, then purified by HPLC (C₁₈, water+0.1% TFA vs acetonitrile). Fractions containing the desired asymmetric dye were identified by TLC/MS, pooled and evaporated under vacuum to low volume and freeze-dried to give the product dye, 6.9 mg. MS (MALDI-TOF): MH⁺=597. UV/VIS (MeOH): ABS λₘₐₓ=488 nm. Fluorescence (MeOH): excitation λₘₐₓ=487 nm; emission λₘₐₓ =504 nm. Accurate mass: MH⁺=C₃₀H₂₇N₂F₂O₇S⁺ requires 597.1507, found MH⁺=597.1510 (0.5 ppm).

### 19. 4-(2-{(1E,3Z)-3-[3-[4-(Carboxymethyl)benzyl]-6-fluoro-1,3-benzoxazol-2(3H)-ylidene]prop-1-enyl}-5,6-difluoro-1,3-benzoxazol-3-ium-3-yl)butane-1-sulfonate (Compound 6)

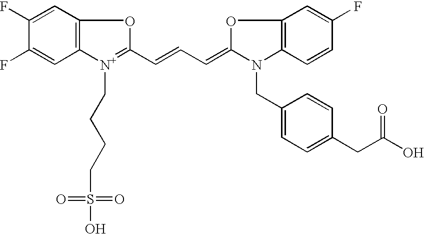

4-(5,6-Difluoro-2-methyl-1,3-benzoxazol-3-ium-3-yl)butane-1-sulfonate (from Example 8, ˜50 mg) and 3-[4-(carboxymethyl)benzyl]-6-fluoro-2-methyl-1,3-benzoxazol-3-ium bromide (from Example 5, ˜50 mg) were dissolved in pyridine (1.5 ml), with warming and sonication. Triethyl orthoformate (0.5 ml) was then added and the resulting mixture heated at 120° C. for 3 hrs to give a deep yellow solution. This was cooled and the solvent evaporated under vacuum. The residue was triturated with diethyl ether, then purified by HPLC (C₁₈, water+0.1% TFA vs acetonitrile). Fractions containing the desired asymmetric dye were identified by TLC/MS, pooled and evaporated under vacuum to low volume and freeze-dried to give the product dye. MS (MALDI-TOF): MH⁺=615. UV/VIS (MeOH): ABS λₘₐₓ=490 nm. Fluorescence (MeOH): excitation λₘₐₓ=490 nm; emission λₘₐₓ=505 nm. Accurate mass: MH⁺=C₃₀H₂₆N₂F₃O₇S⁺requires 615.1413, found MH⁺=615.1421 (1.3 ppm).

### 20. 3-(5-Carboxypentyl)-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 7)

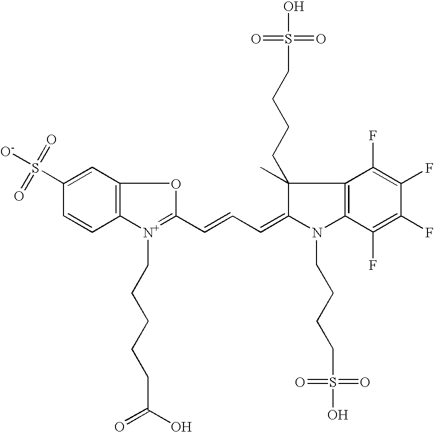

3-(5-Carboxypentyl)-2-methyl-1,3-benzoxazol-3-ium-6-sulfonate (from Example 13.2, 13 mg; 0.04 mmol) was dissolved in acetic acid:acetic anhydride (10:1; 5 ml), then treated with diphenylformamidine (7 mg; 0.037 mmol). After heating at 120° C. for 1 hour, the reaction mixture was treated with 4-[4,5,6,7-tetrafluoro-2,3-dimethyl-3-(4-sulfobutyl)-3H-indolium-1-yl]butane-1-sulfonate (from Example 11, 20 mg; 0.04 mmol) and potassium acetate (40 mg; 0.4 mmol). After heating the reaction mixture at 80° C. for 30 min, the reaction mixture was concentratedin vacuo and the product, 3-(5-carboxypentyl)-6-sulfo-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium, was isolated from the resultant oil by HPLC. MS (MALDI-TOF): MH⁺=827.

### 21. Activation of a carboxy-dye: Preparation of 3-{6-[(2,5-dioxopyrrolidin-1-yl)oxy]-6-oxohexyl}-2-{(1E,3E)-3-[4,5,6 7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 8)

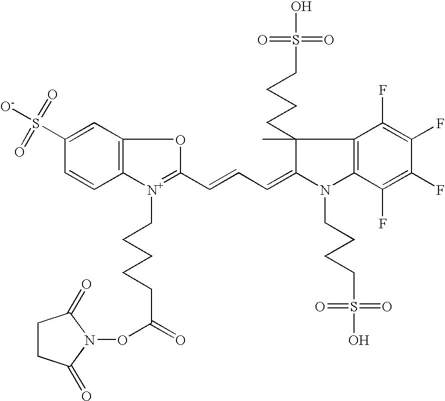

3-(5-Carboxypentyl)-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (from Example 20, 2 mg) was dissolved in DMF (400 μl) containing diisopropylethylamine (1.6 μl; 4% v/v). Dipyrrolidino-(N-succinimidyloxy)-carbenium hexafluorophosphate (10 mg) was added and the solution agitated for 1 hour prior to analysis by TLC. Total conversion to a new spot was observed. The product, 3-{6-[(2,5-dioxopyrrolidin-1-yl)oxy]-6-oxohexyl}-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 8) was used immediately for antibody conjugation.

### 22. Antibody Conjugation

Goat anti-mouse IgG; (Rockland; 0.5 mg in PBS/Azide) was buffer exchanged with NaHCO₃ buffer (0.1 M; pH 9.2) using size exclusion chromatography (Biorad ECONO-PAC™ 10DG Desalting Column). The concentration of the solution was measured by UV spectroscopy. Compound 8 (8 molar equivalents) was dissolved in DMF then added to the IgG solution and stirred at room temperature for 1 h. The reaction mixture was purified by size exclusion chromatography with PBS (0.1 M; pH 7.2). The dye:protein ratio was determined by UV spectroscopy. The product was isolated by freeze drying.

### 23. Photostability Studies

Photostability studies were performed as described below. All fluorophores were dissolved in water and exposed to a strong light source.

A Wallac light box (1295-013) was employed as the strong light source. Samples were maintained at 22 cm above the light source, with continuous exposure to light. The UV/visible spectrum of each sample was measured once every twenty four hours. The same cuvettes and spectrophotometer were used for each measurement point. The following experiment was performed:

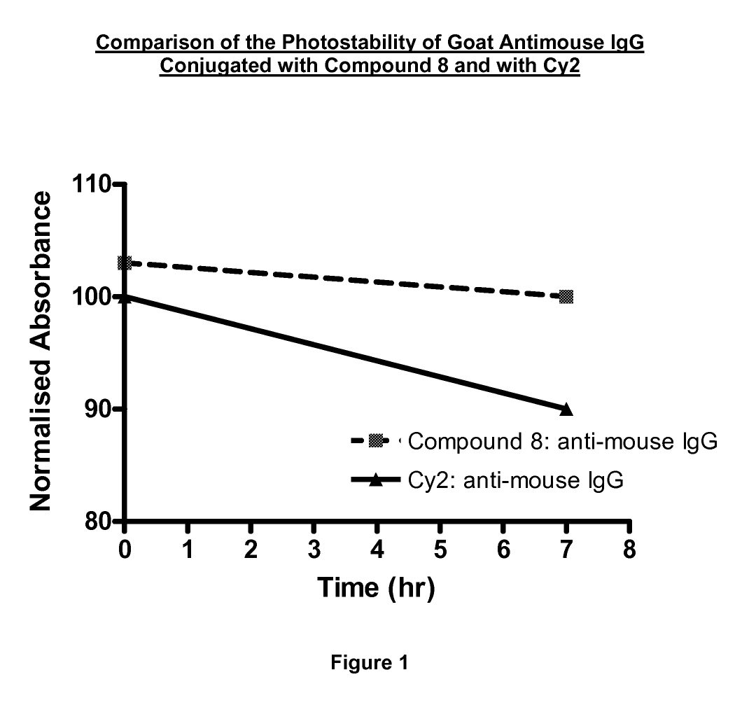

The photostability of the anti-mouse IgG:conjugate with Compound 8 was studied in comparison with a non-fluorinated analogue, CY™2: IgG conjugate. The data for each experiment was normalised and plotted as shown in FIG. 1. The result demonstrates that the fluorinated dye exhibits a greater resistance to photobleaching when compared with the non-fluorinated dye analogue.

### 24. Western Blotting

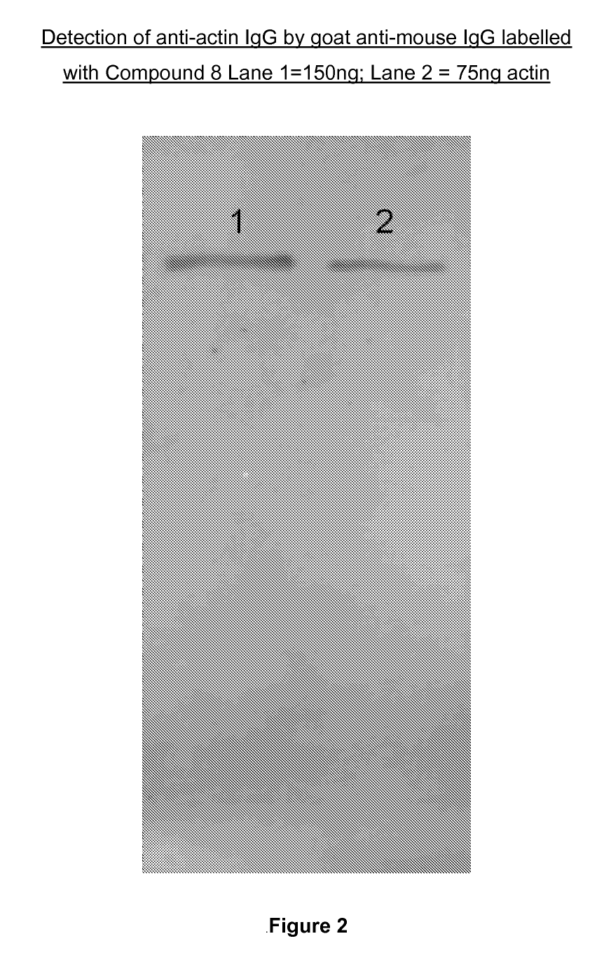

Actin (Sigma; A3653) was diluted with sample loading buffer (0.5M Tris-HCl, SDS, glycerol, bromophenol blue) (SLB;) to form a stock solution of 1 μg/μl. After a further 1 in 30 dilution with SLB, the protein was loaded onto a 12% Tris Glycine gel (Invitrogen NOVEX®; EC60055BOX) gel and run at 100V for approximately 2 hours. After transferring the protein to a HYBOND™ LFP membrane (GE Healthcare) and blocking overnight, the membrane was treated with the primary antibody (Sigma; monoclonal anti-actin; A4700). After a series of wash steps, the membrane was incubated for 1 hour with the goat anti-mouse IgG:Compound 8 conjugate (from Example 22) diluted 1:2500 with wash/block solution (PBS, 0.1 % Tween-20). After another series of wash steps, the membrane was dried at 37° C. for 1 hour then analysed on a TYPHOON™ 8600 (Filter set=Fluorescein; PMT=550V; Pixel size=100 μm). The result is shown in FIG. 2.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1. A compound of formula (I): 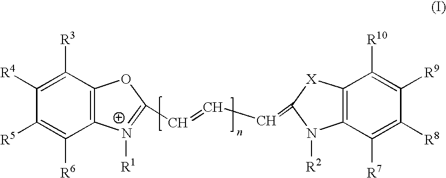
wherein:
X is selected from the group consisting of —O—, —S— and 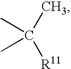
where R¹¹ is CH₃ or —(CH₂)ₖ—SO₃H;
at least one of groups R¹ and R² is the group -L-R^{x} or -L-R^{p}, where L is a linking group having a chain from 1-20 linked atoms selected from the group consisting of carbon, nitrogen and oxygen atoms;
R^{x} is a group suitable for covalent attachment of said compound to a component;
R^{p} is a component;
when either of groups R¹ and R² is not said group -L-R^{x} or -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine.

2. The compound of claim 1, wherein when X is —O— or —S—, groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4.

3. The compound of claim 1, having the formula (II): 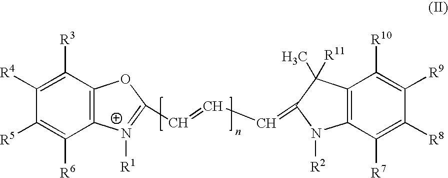
wherein at least one of groups R¹ and R² is the group -L-R^{x} or -L-R^{p}, where L, R^{x} and R^{p} are hereinbefore defined;
when either of groups R¹ and R² is not said group -L-R^{x} or -L-R^{p}, said remaining group R¹ or R² is selected from C₁-C₄ alkyl and —(CH₂)ₖ—SO₃H;
R¹¹ is CH₃ or —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

4. The compound of claim 1 having the formula (III): 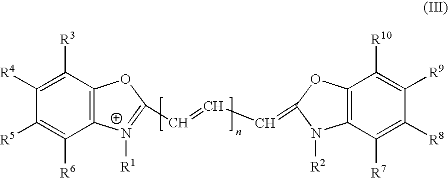
wherein at least one of groups R¹ and R² is the group -L-R^{x} or -L-R^{p}, where L, R^{x} and R^{p} are hereinbefore defined;
when either of groups R¹ and R² is not said group -L-R^{x} or -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4;
k is an integer from 1 to 10 and n is an integer from 1 to 3;
provided that at least one of groups R³, R⁴, R⁵, R⁶, R⁷, R⁸, R⁹ and R¹⁰ comprises fluorine.

5. The compound of claim 1, wherein at least one of groups R³, R⁴, R⁵, R⁶, R⁷, R⁸, R⁹ and R¹⁰ is fluorine.

6. The compound of claim 1, wherein at least two of groups R³, R⁴, R⁵ and R⁶ and/or groups R⁷, R⁸, R⁹ or R¹⁰ are F and any remaining groups R³, R⁴, R⁵, R⁶, R⁷, R⁸, R⁹ or R¹⁰ are selected from H or —SO₃H.

7. The compound of claim 1, wherein groups R³, R⁴, R⁵ and R⁶ and/or groups R⁷, R⁸, R⁹ and R¹⁰ are F.

8. The compound of claim 1, wherein at least one of groups R³, R⁴, R⁵ or R⁶ and/or groups R⁷, R⁸, R⁹ or R¹⁰ are perfluoro C₁-C₄ alkyl and any remaining groups R³, R⁴, R⁵, R⁶, R⁷, R⁸, R⁹ and R¹⁰ are selected from H or F.

9. The compound of claim 8, wherein not more than two of the R³, R⁴, R⁵ or R⁶ positions and/or the R⁷, R⁸, R⁹ or R¹⁰ positions are substituted by perfluoro C₁-C₄ alkyl.

10. The compound of claim 8, wherein said perfluoro C₁-C₄ alkyl is trifluoromethyl.

11. The compound of claim 1, wherein —(CH₂)ₖ—SO₃H is selected from —(CH₂)₃—SO₃H and —(CH₂)₄—SO₃H.

12. The compound of claim 1, wherein group R^{x} comprises a reactive group for reaction with a functional group on a target material, or a functional group for reaction with a reactive group on a target material.

13. The compound of claim 12, wherein said reactive group is selected from the group consisting of succinimidyl ester, sulpho-succinimidyl ester, isothiocyanate, maleimide, haloacetamide, acid halide, hydrazide, vinylsulphone, dichlorotriazine and phosphoramidite.

14. The compound of claim 12, wherein said functional group is selected from the group consisting of hydroxy, amino, sulphydryl, imidazole, carbonyl including aldehyde and ketone, carboxylic acid and thiophosphate.

15. The compound of claim 1, wherein said group R^{x} comprises an affinity tag.

16. The compound of claim 1, wherein said linking group L is selected from linear or branched C₁₋₂₀ alkyl chains, which may optionally contain one or more linkages selected from —O—, —NR′—, —C(O)—NR′— and phenylene, where R′ is hydrogen or C₁-C₄ alkyl.

17. The compound of claim 16, wherein L has from 5 to 12 atoms.

18. The compound of claim 16, wherein L is the group —(CH₂)ₚ-Q-(CH₂)ᵣ— where Q is selected from: —CH₂— and —CO—NH—, p is 1-5 and r is 0-5.

19. A compound selected from:
i) 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate (Compound 1);
ii) 4-(2-{(1E,3E)-3-[1-(5-Carboxypentyl)-5,7-difluoro-3-methyl-3-(4-sulfobutyl)-1,3-dihydro-2H-indol-2-ylidene]prop-1-enyl}-6-fluoro-1,3-benzoxazol-3-ium-3-yl)butane-1-sulfonate (Compound 2);
iii) 4-(2-{(1E,3E)-3-[1-(5-Carboxypentyl)-4,5,6,7-tetrafluoro-3-methyl-3-(4-sulfobutyl)-1,3-dihydro-2H-indol-2-ylidene]prop-1-enyl}-5-fluoro-1,3-benzoxazol-3-ium-3-yl)butane-1-sulfonate (Compound 3);
iv) 4-[(2E)-2-((2E)-3-{3-[4-(Carboxymethyl)benzyl]-6-fluoro-1,3-benzoxazol-3-ium-2-yl}prop-2-enylidene)-4,5,6,7-tetrafluoro-3-methyl-3-(4-sulfobutyl)-2,3-dihydro-1H-indol-1-yl]butane-1-sulfonate (Compound 4);
v) 4-(2-{(1E,3Z)-3-[3-[4-(Carboxymethyl)benzyl]-6-fluoro-1,3-benzoxazol-2(3H)-ylidene]prop-1-enyl}-6-fluoro-1,3-benzoxazol-3-ium-3-yl)butane-1-sulfonate (Compound 5);
vi) 4-(2-{(1E,3Z)-3-[3-[4-(Carboxymethyl)benzyl]-6-fluoro-1,3-benzoxazol-2(3H)-ylidene]prop-1-enyl}-5,6-difluoro-1,3-benzoxazol-3-ium-3-yl)butane-1-sulfonate (Compound 6);
vii) 3-(5-Carboxypentyl)-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 7); and
viii) 3-{6-[(2,5-Dioxopyrrolidin-1-yl)oxy]-6-oxohexyl}-2-{(1E,3E)-3-[4,5,6,7-tetrafluoro-3-methyl-1,3-bis(4-sulfobutyl)-1,3-dihydro-2H-indol-2-ylidene]prop-1-enyl}-1,3-benzoxazol-3-ium-6-sulfonate (Compound 8).

20. A method for labelling a component, the method comprising:
i) contacting said component with a compound of formula (I): 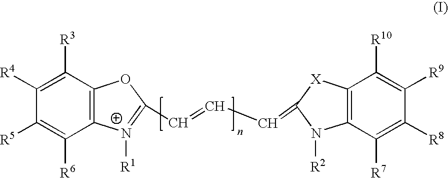
wherein:
X is selected from the group consisting of —O—, —S— and 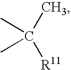
where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine; and
ii) incubating said compound with said component under conditions suitable for binding to and thereby labelling said component.

21-27. (canceled)

28. A fluorescently-labelled dye conjugate of a component having the formula (I): 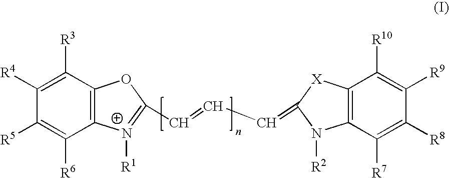
wherein:
X is selected from the group consisting of —O—, —S— and 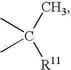
where R¹¹ is CH₃ or —(CH₂)ₖ—SO₃H;
at least one of groups R¹ and R² is the group -L-R^{p}, where L is a linking group having a chain from 1-20 linked atoms selected from the group consisting of carbon, nitrogen and oxygen atoms;
R^{p} is a component;
when either of groups R¹ and R² is not said group -L-R^{p}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine.

29-33. (canceled)

34. A pharmaceutical composition which comprises the dye conjugate of claim 28 together with a biocompatible carrier, in a form suitable for mammalian administration.

35. The pharmaceutical composition of claim 34, wherein the fluorescent dye is conjugated to a component comprising a BTM as defined hereinbefore.

36. (canceled)

37. A method of in vivo optical imaging of the mammalian body which comprises use of the dye conjugate of claim 34 to obtain images of sites of localisation of the BTM in vivo.

38. (canceled)

39. A method for detecting a secondary component in a sample comprising the steps of:
i) contacting a sample containing or suspected to contain the secondary component to be detected with a primary component under conditions to form a complementary specific binding pair and wherein said primary component is labelled with a compound of formula (I): 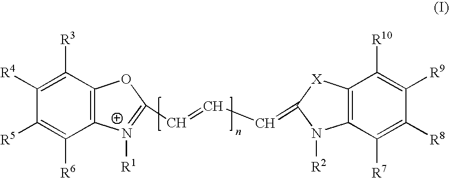
wherein:
X is selected from the group consisting of —O—, —S— and 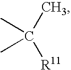
where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined;
k is an integer from 1 to 10 and n is an integer from 1 to 3;
provided that at least one of groups R³, R⁴, R⁵, R⁶, R⁷, R⁸, R⁹ and R¹⁰ comprises fluorine;
ii) binding said labelled primary component to said second component to form a labelled secondary component; and
iii) detecting said labelled secondary component by an optical method.

40. (canceled)

41. A process comprising:
a) reacting a first compound having the formula (A): 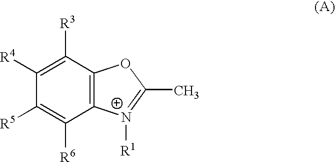
with
b) a second compound which may be the same or different from the first compound and having the formula (B): 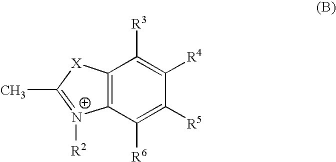
and
c) a third compound (C) suitable for forming a conjugated linkage between said first and second compounds;
wherein:
X is selected from the group consisting of —O—, —S— and 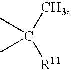
where R¹¹ is CH₃ or —(CH₂)ₖ—SO₃H;
at least one of groups R¹ and R² is the group -L-R^{x}, where L is a linking group having a chain from 1-20 linked atoms selected from the group consisting of carbon, nitrogen and oxygen atoms;
R^{x} is a group suitable for covalent attachment of said compound to a component;
when either of groups R¹ and R² is not said group -L-R^{x}, said remaining group R¹ or R² is selected from C₁-C₄ alkyl and —(CH₂)ₖ—SO₃H;
groups R³, R⁴, R⁵, R⁶, R⁷, R⁸, R⁹ and R¹⁰ are selected independently from hydrogen, —SO₃H and the group —(CF₂)ₘ—F, where m is 0 or an integer from 1 to 4; or R³ taken in combination with R⁴ or R⁵ taken in combination with R⁶ and/or R⁷ taken in combination with R⁸ or R⁹ taken in combination with R¹⁰ form a fused aromatic six-membered ring containing carbon atoms which may be optionally substituted one or more times by —SO₃H or —(CF₂)ₘ—F, where m is hereinbefore defined; and
k is an integer from 1 to 10;
provided that at least one of groups R³, R⁴, R⁵, R⁶, R⁷, R⁸, R⁹ and R¹⁰ comprises fluorine.

42. A compound of formula (A): 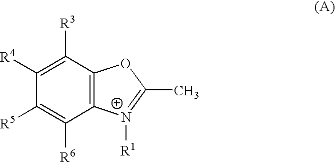
wherein:
R¹ is selected from —(CH₂)ₖ—SO₃H, -L-R^{x} and -L-R^{p} where L is a linking group having a chain from 1-20 linked atoms selected from the group consisting of carbon, nitrogen and oxygen atoms;
R^{x} is a group suitable for covalent attachment of said compound to a component;
R^{p} is a component;
groups R³, R⁴, R⁵ and R⁶ are selected independently from hydrogen, —SO₃H and the group —(CF₂)ₘ—F, where m is 0 or an integer from 1 to 4;
provided that at least one of groups R³, R⁴, R⁵ and R⁶ comprises fluorine.
